(19) United States
(12) Patent Application Publication
(10) Pub. No.: US 2018/0055878 A1
(43) Pub. Date: Mar. 1, 2018

(54) TOPICAL ARTHRITIS TREATMENT DEVICE

(71) Applicants: Jeffrey P. Hendricks, Columbus, NC (US); Teresa Hendricks, Grand Rapids, MI (US)

(72) Inventors: Jeffrey P. Hendricks, Columbus, NC (US); Teresa Hendricks, Grand Rapids, MI (US)

(21) Appl. No.: 15/690,653

(22) Filed: Aug. 30, 2017

Related U.S. Application Data
(60) Provisional application No. 62/382,481, filed on Sep. 1, 2016.

(51) Int. Cl. A61K 33/24; A61K 9/00

(52) U.S. Cl. CPC A61K 33/24; A61K 9/0014

(57) ABSTRACT

The present disclosure relates to a method of forming a topical treatment device for arthritis. The method can include determining an arthritis treatment surface area of a user, forming a flexible metal substrate conforming to the arthritis treatment surface area, and forming a gold composition on the flexible metal substrate.

### CROSS-REFERENCE TO RELATED APPLICATION

This application claims the benefit of U.S. Provisional Patent Application No. 62/382,481, filed Sep. 1, 2016, which is incorporated herein by reference in its entirety.

### BACKGROUND

Rheumatoid arthritis and other auto-immune arthritis are long-term autoimmune disorders that typically result in warm, swollen, and painful joints. Rheumatoid arthritis affects the lining of joints and can eventually result in bone erosion and joint deformity. Furthermore, rheumatoid arthritis also can damage a wide variety of body systems, including the skin, eyes, lungs, heart and blood vessels. Treatment of rheumatoid arthritis involves drugs to reduce pain and inflammation in attempts to improve a person's overall functioning.

### BRIEF SUMMARY

In one aspect, the present disclosure relates to a topical treatment device for arthritis including a flexible metal substrate conformed to a user treatment surface area, and at least one gold-containing composition formed on the metal substrate.

In another aspect, the present disclosure relates to a method of forming a topical treatment device for arthritis, the method including determining an arthritis treatment surface area of a user, forming a flexible metal substrate conforming to the arthritis treatment surface area, and forming a gold composition on the flexible metal substrate.

### BRIEF DESCRIPTION OF THE DRAWINGS

In the drawings:

FIG. 1 is a schematic view of a treatment surface area and imaging device according to aspects described herein.

FIG. 2 is an isometric view of a topical treatment device according to aspects described herein.

FIG. 3 is a cross-sectional view of the topical treatment device along line of FIG. 2 according to aspects described herein.

FIG. 4 is a flow chart of a method of forming a topical treatment device according to aspects described herein.

### DETAILED DESCRIPTION

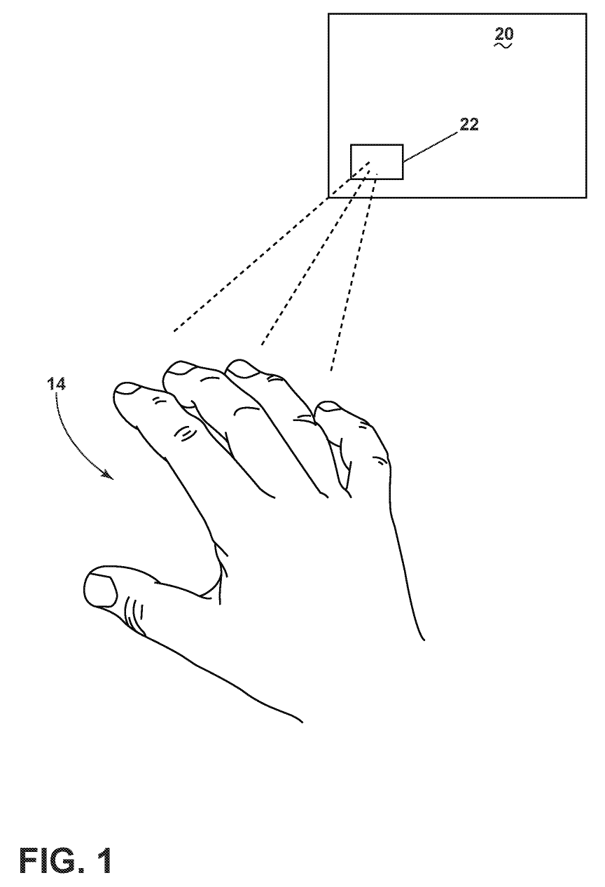

FIG. 1 is a schematic view of a user treatment surface area 14 and imaging device 20 according to aspects described herein. A user treatment surface area 14 can be any body part that a user wishes to treat such as a hand 14, as shown in FIG. 1, foot, shoulder, and the like. In non-limiting aspects, the user treatment surface area 14 can include a limited portion of a body part (e.g. a subset of fingers, a heel, wrist, etc.). An imaging device 20 can determine the user treatment surface area 14 by way of magnetic resonance imaging, ionizing radiation (including but not limited to CT scans, X-rays, fluoroscopy, or the like), ultrasound, bone densitometry, three-dimensional imaging, visual imaging, plaster mold casting, foam mold casting, or direct measurement. In this sense, “determining” the user treatment surface area 14 can include scanning, imaging, evaluating, or generally observing a portion of a body part intended to be topically treated, as described herein.

An imaging device 20 can include a sensor 22 configured to detect, measure, scan, evaluate, observe, the like, or a combination thereof, the user treatment surface area 14. While sensors can be described as “sensing” or “measuring” a respective value, sensing or measuring can include determining a value indicative of or related to the respective value, rather than directly sensing or measuring the value itself. The sensed or measured values can further be provided to additional components. For instance, the value can be provided to a controller module or processor, and the controller module or processor can perform processing on the value to determine a representative value or an electrical characteristic representative of said value.

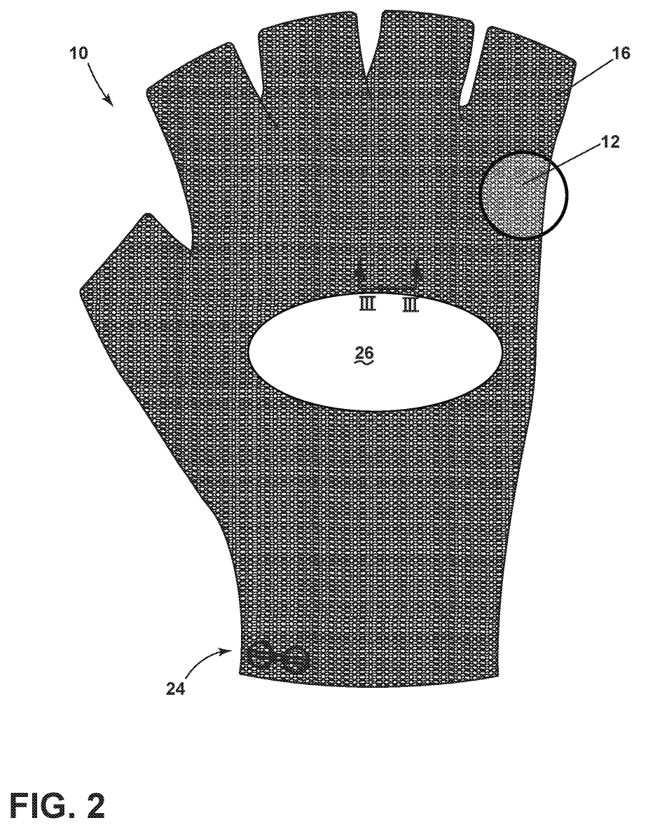

FIG. 2 is a perspective view of a topical treatment device 10 according to aspects described herein. The topical treatment device 10 can be tailored, conformed, shaped, or otherwise formed to fit the user treatment surface area 14 such that the topical treatment device 10 touches, contacts, or otherwise physically interacts with the user treatment surface area 14. For example, a topical treatment device 10 can be in the form of a glove (as shown) that can be fit to conform to a user's hand in order for a user to wear the topical treatment device 10. The topical treatment device 10 includes a flexible metal substrate 12, which is shown in the circular cut-away portion of FIG. 2. The flexible metal substrate 12 can be in the form of chainmail, which can be made of a plurality of linked, metal rings. Non-limiting example compositions of the metal rings can include stainless steel, surgical stainless steel, or any suitable metal alloy. The pattern of the linked rings can vary depending on the user treatment surface area 14, desired flexibility, desired conforming, or a combination thereof. For example, in the case that the user treatment surface area 14 is a shoulder, the linked rings may be larger or more spread apart than the linked rings for a flexible metal substrate 12 for a hand. The conforming of the topical treatment device 10 can be related to or based on the desired flexibility of the flexible metal substrate 12, or the expected movement of the underlying user treatment surface area 14. For example, a user treatment surface area 14 for a set of fingers can require increased flexibility or modified conformity compared with a user treatment surface area 14 for a forearm. Modifications to conformity or flexibility can be implemented or included after determining the user treatment surface area 14.

A gold-containing layer 16 can further be formed on the flexible metal substrate 12. In one non-limiting example, the gold-containing layer 16 can be plated on the flexible metal substrate 12 such that a thin coating of gold or a gold-containing composition is deposited onto the surface of the flexible metal substrate 12 such that the gold-containing layer 16 is in flexible and topical contact with the user treatment surface area 14. Plating can be achieved by chemical methods, electrochemical methods, such as barrel plating, or any other methods suitable for depositing gold onto the surface of the flexible metal substrate 12. The plated gold-containing layer 16 can be at least 14-Karat gold and can have a hardness with HK values in the range of about 200-400. HK values are a result of the Knoop harness test, which is a test for mechanical harness for materials. In one non-limiting example, aspects of the disclosure can be included wherein the plating of the gold-containing layer 16 does not affect the conforming or the flexibility of the underlying flexible metal substrate 12, or affects the same within a tolerance level.

Further non-limiting examples of the topical treatment device 10 can include fasteners, such as a hook, snap, elastic material, magnetic connector, clamp, zipper, tie, clasp 24, or the like. In the illustrated example, the clasp 24 can be fastened so that the topical treatment device 10 fits securely to, at, or with the user treatment surface area 14, while in topical application to the user treatment surface area 14. The fastener can be in the form of any suitable fastener including, but not limited to: a toggle, a fishhook, an “S” hook, a hook-and-eye, a lobster claw, or a springring. Furthermore, the topical treatment device 10 can include gaps, breaks, openings, or an aperture 26. The aperture 26 can be provided, located, disposed, arranged, or the like based on considerations including, but not limited to, increasing flexibility of the topical treatment device 10, reducing the amount of material required to form the topical treatment device 10, or to avoid or exclude a portion of the user treatment surface area 14, such as were treatment is not needed or not desirable.

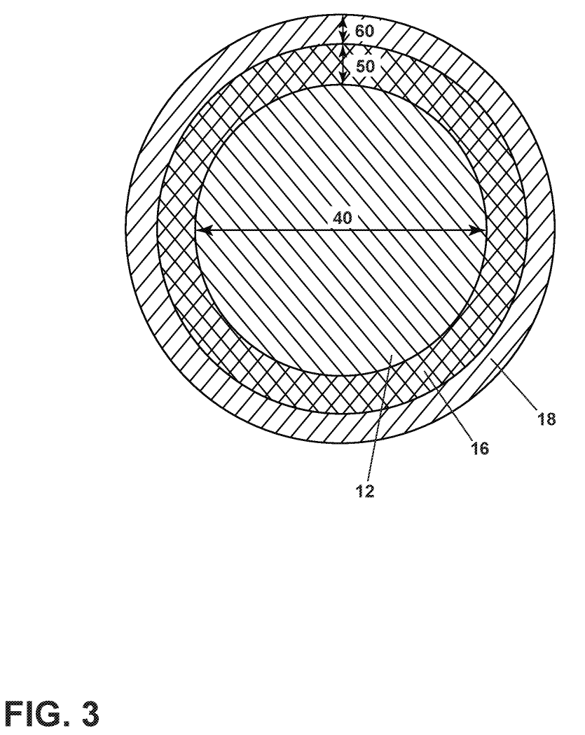

Turning to FIG. 3, a cross-section of the topical treatment device 10 along line of FIG. 2 is illustrated. The flexible metal substrate 12 can include a set of gold-containing layers, such as a first gold-containing layer 16 and a second gold-containing layer 18. The first gold-containing layer 16 can be formed on the flexible metal substrate 12, while the second gold-containing layer 18 can be formed on the first gold-containing layer 16. The second gold-containing layer 18 composition can be different from the first gold-containing layer 16 composition. For example, the second gold-containing layer 18 composition can be pure gold, or 24-Karat gold, while the first gold-containing layer 16 composition can be 14-Karat gold. As used herein, a “Karat” is a measure of 1/24th of composition part by weight. However, the first and second gold-containing layers 16, 18 can have any suitable amount, or part by weight, of gold, including equal amounts. At least one of the first or second gold-containing layers 16, 18 can have a hardness with HK values in the range of about 200-400.

The flexible metal substrate 12, the first gold-containing layer 16, and the second gold-containing layer 18 can include various thicknesses. The flexible metal substrate 12 can have the largest thickness 40, with thicknesses around 3 millimeters or more. The first gold-containing layer 16 can have a much smaller thickness 50, with thicknesses in the range of between 4-8 micrometers. The second gold-containing layer 18 can have an even smaller thickness 60, with thicknesses in the range of between 2-4 micrometers. The first and second gold-containing layers 16, 18 can each have thicknesses 50, 60, up to about 8 micrometers due to plating techniques.

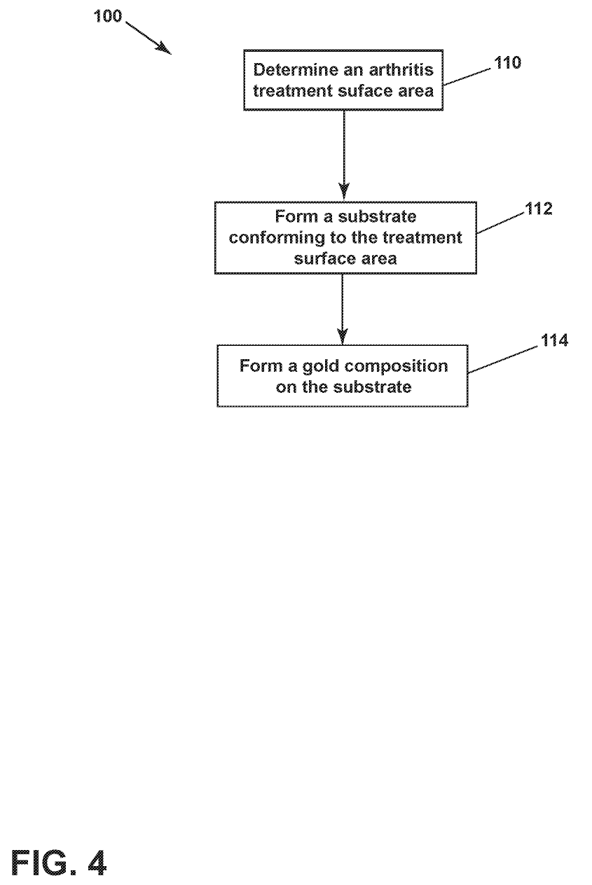

FIG. 4 illustrates a method 100 of forming the topical treatment device 10 for arthritis. The method 100 can start with determining an arthritis treatment surface area 14 of a user, which can include imaging a body part 110 with the imaging device 20, at 110. Furthermore, determining can include at least one of three-dimensional imaging, visual imaging, plaster mold casting, foam mold casting, or direct measurement of the arthritis treatment surface area. Then, the flexible metal substrate 12 can be formed to conform to the arthritis treatment surface area 14 of the user, which can include shaping a chainmail substrate tailored to the body part, at 112. Next, a gold composition can be formed on the flexible metal substrate 12, which can include forming a layer of gold composition on the chainmail substrate, at 114. Furthermore, forming a gold composition on the flexible metal substrate 12 can include plating a first gold composition 16 on the flexible metal substrate and plating a second gold composition 18 on the first gold composition 16.

Lastly, the method 100 can include applying the topical treatment device 10 to the arthritis treatment area 14, for example, by wearing the topical treatment device 10 on the user treatment surface area 14. Preferably, the user can apply the topical treatment device 10 to the arthritis treatment area 14 while the user is sleeping, or for about 8-10 hours during the day.

The sequence depicted is for illustrative purposes only and is not meant to limit the method 100 in any way as it is understood that the portions of the method can proceed in a different logical order, additional or intervening portions can be included, or described portions of the method can be divided into multiple portions, or described portions of the method can be omitted without detracting from the described method.

One beneficial and advantageous feature of the present disclosure is a topical application of gold for treatment of arthritis. It has been observed that patients suffering from arthritis wearing a gold band on a finger can exhibit normal joint conditions in the finger having the gold band, while joints in fingers without a gold band exhibit symptoms of arthritis, such as swelling and deformity. While not wishing to be bound to theory, it is believed that fingers having a gold band slowly absorb gold from the band and that the gold is distributed towards the distal end of the finger through the lymphatic system. Since the concentration of gold is very low, the gold does not recirculate any further in the lymphatic system and therefore does not affect other joints within the body.

To the extent not already described, the different features and structures of the various embodiments may be used in combination with each other as desired. That one feature may not be illustrated in all of the embodiments is not meant to be construed that it cannot be, but is done for brevity of description. Thus, the various features of the different embodiments may be mixed and matched as desired to form new embodiments, whether or not the new embodiments are expressly described.

While the invention has been specifically described in connection with certain specific embodiments thereof, it is to be understood that this is by way of illustration and not of limitation. Reasonable variation and modification are possible within the scope of the forgoing disclosure and drawings without departing from the spirit of the invention which is defined in the appended claims.

What is claimed is:

1. A topical treatment device for arthritis comprising:
a flexible metal substrate conformed to a user treatment surface area; and
at least one gold-containing composition formed on the metal substrate.

2. The topical treatment device of claim 1 wherein the flexible metal substrate is chainmail.

3. The topical treatment device of claim 2 wherein the chainmail is steel.

4. The topical treatment device of claim 1 comprising a first gold-containing composition formed on the metal substrate and a second gold-containing composition formed on the first gold-containing composition.

5. The topical treatment device of claim 4 wherein the second gold-containing composition is 24 karat gold.

6. The topical treatment device of claim 4 wherein at least one of the first or second gold-containing compositions comprises at least 14 karat gold.

7. The topical treatment device of claim 4 wherein the gold composition of the first gold-containing composition is different from the gold composition of the second gold-containing composition.

8. The topical treatment device of claim 4 wherein at least one of the first or second gold-containing composition is formed by plating.

9. The topical treatment device of claim 8 wherein at least one of the first or second gold-containing composition is formed by a barrel plating process.

10. The topical treatment device of claim 4 wherein the first gold-containing composition has a hardness of 200 HK.

11. The topical treatment device of claim 4 wherein the second gold-containing composition has a hardness between 200 and 400 HK.

12. The topical treatment device of claim 1 wherein the flexible metal substrate is tailored to fit the user treatment surface area.

13. The topical treatment device of claim 1 wherein the flexible metal substrate is conformed to the user treatment surface area such that the at least one gold-containing composition is in flexible and topical contact with the user treatment surface area.

14. The topical treatment device of claim 1 wherein the at least one gold-containing composition comprises at least 14 karat gold.

15. The topical treatment device of claim 1 wherein the at least one gold-containing composition has a hardness of 200 HK.

16. A method of forming a topical treatment device for arthritis, the method comprising:
determining an arthritis treatment surface area of a user;
forming a flexible metal substrate conforming to the arthritis treatment surface area; and
forming a gold composition on the flexible metal substrate.

17. The method of claim 16 wherein determining includes at least one of three-dimensional imaging, visual imaging, plaster mold casting, foam mold casting, or direct measurement of the arthritis treatment surface area.

18. The method of claim 16 wherein the forming the flexible metal substrate includes forming the flexible metal substrate from chainmail.

19. The method of claim 16 wherein the forming a gold composition on the flexible metal substrate further includes plating a first gold composition on the flexible metal substrate and plating a second gold composition on the first gold composition.

20. The method of claim 16 further comprising applying the topical treatment device to the arthritis treatment surface area.
